(19) United States
(12) Patent Application Publication
(10) Pub. No.: US 2010/0184067 A1
(43) Pub. Date: Jul. 22, 2010

(54) PRIMER EVALUATION METHOD, PRIMER EVALUATION PROGRAM, AND REAL-TIME POLYMERASE CHAIN REACTION APPARATUS

(75) Inventors: Tomoteru Abe, Tokyo (JP); Kazuhiro Nakagawa, Saitama (JP); Satomi Tanaka, Tokyo (JP)

Correspondence Address: K&L Gates LLP, P. O. BOX 1135, CHICAGO, IL, 60690, US

(73) Assignee: SONY CORPORATION, Tokyo (JP)

(21) Appl. No.: 12/686,852

(22) Filed: Jan. 13, 2010

(30) Foreign Application Priority Data
Jan. 20, 2009 (JP) 2009-009984

(51) Int. Cl. C12Q 1/68; C12M 1/34

(52) U.S. Cl. 435/6; 435/287.2

(57) ABSTRACT

A primer evaluation method includes: acquiring a signal indicating time change in an amplification amount obtained when sample sets prepared for the number of temperature conditions that should be made different from each other at an annealing stage in units of target nucleic acids diluted in a stepwise manner are so amplified that a temperature condition at a stage other than the annealing stage is fixed; acquiring a signal indicating initial amounts of the target nucleic acids diluted in a stepwise manner; obtaining amplification efficiency for each of the temperature conditions based on the time change in the amplification amount and the initial amount, and calculating a variation degree of the amplification efficiency; and submitting the variation degree and a reference value for quality evaluation of a primer, set with respect to the variation degree.

### CROSS REFERENCES TO RELATED APPLICATIONS

The present application claims priority to Japanese Priority Patent Application JP 2009-009984 filed in the Japan Patent Office on Jan. 20, 2009, the entire content of which is hereby incorporated by reference.

### BACKGROUND

The present disclosure relates to a primer evaluation method, a primer evaluation program, and a real-time PCR apparatus and is suitable in e.g. the technical field to amplify the nucleic acid.

In the polymerase chain reaction (PCR), a deoxyribonucleic acid (DNA) as the amplification subject, a DNA synthetase, and a large amount of primers are mixed. The primer is a short nucleic acid fragment taking a role to supply the DNA polymerase with the start point of the DNA synthesis reaction and is essential in any DNA synthesis reaction. Therefore, the design of the primer is important.

For the design of the primer, there has been proposed a method in which the plural sequences that should be employed as the candidates are synthesized and screened to obtain the optimum sequence satisfying predetermined conditions (refer to e.g. Japanese Patent Laid-open No. 2003-210175 and Japanese Patent Laid-open No. 2001-258576).

However, in this design method, although the base length and the GC content or the Tm value are taken into consideration as the predetermined conditions, change in the amplification efficiency due to change in the reaction temperature is not taken into consideration. The reason for this will be because this method is based on the premise that the temperature condition at the annealing stage, at which the primer should function, is set to about 60° C.

However, in the case of actually amplifying a DNA by using a real-time PCR apparatus or a PCR apparatus, the difference in the temperature condition attributed to the aging deterioration of the PCR apparatus, change in the temperature outside the PCR apparatus, and so on inevitably exists. Furthermore, in recent years, portable PCR apparatus has been proposed by the present assignee, responding to demands for apparatus size reduction. Thus, the use of the PCR apparatus under environments in which the difference in the temperature condition occurs at a higher degree is possible.

Therefore, even with a primer designed by the above-described design method, the amplification efficiency of the PCR products (nucleic acid) changes depending on the temperature in the actual use of the PCR apparatus. Even with a primer that is currently available commercially, a similar result is caused regarding the change in the amplification efficiency of the nucleic acid attributed to the temperature.

The change in the amplification efficiency of the nucleic acid due to temperature change is equivalent to change in the quantitative value of the nucleic acid as the amplification subject. Therefore, e.g. in the case of monitoring the amount of nucleic acid in a certain living substance every predetermined period, the amplification efficiency of the nucleic acid changes due to the difference among the temperatures at the respective quantitation timings, and thus the value of comparison of the quantified nucleic acid is reduced. This is a particularly serious issue in clinical practice. Consequently, a primer that exhibits amplification efficiency higher than a certain value irrespective of temperature change is required.

There is therefore a need to propose a primer evaluation method, a primer evaluation program, and a real-time PCR apparatus that enable the design of a primer having temperature resistance at a certain level.

### SUMMARY

According to an embodiment, there is provided a primer evaluation method including the steps of: acquiring a signal indicating time change in an amplification amount obtained when sample sets prepared for the number of temperature conditions that should be made different from each other at an annealing stage in units of target nucleic acids diluted in a stepwise manner are so amplified that a temperature condition at a stage other than the annealing stage is fixed; acquiring a signal indicating the initial amounts of the target nucleic acids diluted in a stepwise manner; obtaining amplification efficiency for each of the temperature conditions based on the time change in the amplification amount and the initial amount, and calculating the variation degree of the amplification efficiency; and submitting the variation degree and a reference value for quality evaluation of a primer, set with respect to the variation degree.

According to another embodiment, there is provided a primer evaluation program causing a computer to execute: acquiring, from apparatus capable of propagating a nucleic acid or a storage medium, a signal indicating time change in an amplification amount obtained when sample sets prepared for the number of temperature conditions that should be made different from each other at an annealing stage in units of target nucleic acids diluted in a stepwise manner are so amplified that a temperature condition at a stage other than the annealing stage is fixed; acquiring a signal indicating the initial amounts of the target nucleic acids diluted in a stepwise manner; obtaining amplification efficiency for each of the temperature conditions based on the time change in the amplification amount and the initial amount, and calculating the variation degree of the amplification efficiency; and submitting the variation degree and a reference value for quality evaluation of a primer, set with respect to the variation degree.

According to a further embodiment, there is provided a real-time PCR apparatus including: a heat source device allocated to a plurality of containers used as a place of amplification reaction of a nucleic acid; a controlling section configured to individually control an amount of heat of the heat source device depending on a temperature set for a corresponding container; and a deciding section configured to decide, for each of sample sets prepared for the number of temperature conditions that should be made different from each other in units of target nucleic acids diluted in a stepwise manner, an annealing-stage temperature that should be set for the containers in which the target nucleic acids in the sample set are disposed. The real-time PCR apparatus further includes: an initial amount acquiring section configured to acquire a signal indicating the initial amounts of the target nucleic acids diluted in a stepwise manner; an amplification amount acquiring section configured to acquire, from a plurality of light receiving devices allocated to the containers, a signal indicating an amplification amount obtained when the sample sets are so amplified that a temperature condition at a stage other than an annealing stage is fixed; a calculating section configured to obtain amplification efficiency for each of the temperature conditions based on time change in the amplification amount and the initial amount, and calculate the variation degree of the amplification efficiency; and a submitting section configured to submit the variation degree and a reference value for quality evaluation of a primer, set with respect to the variation degree.

According to the embodiments of the present invention, the temperature dependence of a primer can be detected because the variation degree of the amplification efficiency obtained when the sample sets prepared for the number of temperature conditions that should be made different from each other at the annealing stage are so amplified that the temperature condition at the stage other than the annealing stage is fixed is obtained. In addition, a primer having temperature resistance at a certain level can be designed because this variation degree is submitted in addition to the reference value for quality evaluation of a primer, set with respect to the variation degree.

Additional features and advantages are described herein, and will be apparent from the following Detailed Description and the figures.

### BRIEF DESCRIPTION OF THE FIGURES

FIG. 1 is a diagram schematically showing the configuration of a real-time PCR apparatus;

FIG. 2 is a diagram showing calibration periods;

FIG. 3 is a diagram schematically showing an arrangement example of standard samples for obtaining amplification curves;

FIG. 4 is a diagram schematically showing the configuration of a primer evaluation index submitter;

FIGS. 5A and 5B are graphs showing experimental result (1);

FIGS. 6A and 6B are graphs showing experimental result (2);

FIGS. 7A and 7B are graphs showing experimental result (3);

FIGS. 8A and 8B are graphs showing experimental result (4);

FIGS. 9A and 9B are graphs showing experimental result (5);

FIG. 10 is a graph showing the variation degrees of the amplification efficiency;

FIG. 11 is flowchart showing the procedure of primer evaluation index submission processing; and

FIG. 12 is a diagram schematically showing the configuration of a real-time PCR apparatus according to another embodiment of the present invention.

### DETAILED DESCRIPTION

Embodiments will be described below. The description will be made in the following order.

<1. Embodiment>

[1-1. Configuration of Real-time PCR Apparatus]

[1-2. Configuration of Primer Evaluation Index Submitter]

[1-3. Procedure of Primer Evaluation Index Submission Processing]

[1-4. Advantageous Effects and so on]

<2. Other Embodiments>

### 1. Embodiment

[1-1. Configuration of Real-Time PCR Apparatus]

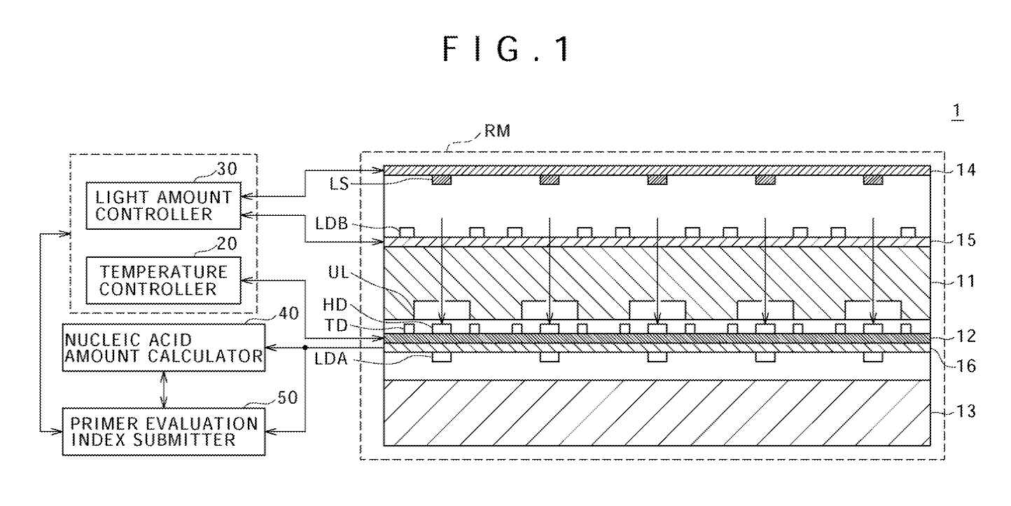

In FIG. 1, the schematic configuration of a real-time PCR apparatus 1 is shown. This real-time PCR apparatus 1 has a structure obtained by disposing plural substrates 11 to 16 in a laminar manner with predetermined intervals for a reaction room RM.

The reaction substrate 11 is a substrate serving as the basic layer. In this substrate, containers (hereinafter, referred to also as wells) UL each used as the place of amplification reaction of a nucleic acid are formed with high density. To these wells UL, the target nucleic acid as the amplification subject and various substances necessary for the amplification of the target nucleic acid (primer, buffer solution, enzyme, dNTP, fluorescent dye, and so on) are given.

For example, if the reaction substrate 11 with a size of six centimeters square is used, about 40000 wells UL each having a volume of 1 μL or lower can be formed. Therefore, this real-time PCR apparatus 1 can deal with the same kind or different kinds of many target nucleic acids even if the size of the reaction room RM is reduced.

The heating substrate 12 is a substrate disposed as a layer on the lower side of the reaction substrate 11. To the surface facing the reaction substrate 11, of the heating substrate 12, heat source devices HD are allocated for each well UL. Plural thermosensing devices TD are disposed around these heat source devices HD. As the heat source device HD, e.g. a thin film transistor (TFT) is used. As the thermosensing device TD, e.g. a PIN diode is used.

A temperature controller 20 is connected to the heat source devices HD and the thermosensing devices TD on the heating substrate 12. The temperature controller 20 individually controls the amount of heat of each heat source device HD depending on the temperature sensed by using the thermosensing devices TD surrounding the heat source device HD at every predetermined interval.

Specifically, the temperature controller 20 sets the target temperature for each well UL for each of the amplification stages (denaturation stage, annealing stage, elongation stage). Furthermore, the temperature controller 20 gives current or voltage with the value dependent on the target temperature to the heat source device HD of the corresponding well UL to thereby heat the respective wells UL.

In addition, every time the temperature is sensed by using the thermosensing device TD at every predetermined interval, the temperature controller 20 obtains the difference between the temperature sensed by the thermosensing device TD and the target temperature and varies the current or voltage given to the heat source device HD depending on the difference for each well UL.

Due to this feature, even if the temperature condition necessary for the amplification differs, this real-time PCR apparatus 1 can adjust the temperature of each of the wells UL disposed with high density with high accuracy for each amplification stage. Therefore, this real-time PCR apparatus 1 can reduce the error rate of the amplification reaction result attributed to the temperature and can enhance the detection accuracy.

The heating assist substrate 13 is a substrate disposed as a layer on the lower side of the heating substrate 12. This heating assist substrate 13 absorbs or radiates the heat of the whole of the reaction room RM to thereby keep the reaction room RM at the prescribed temperature.

Therefore, this real-time PCR apparatus 1 can set a short time period as the time period until the temperature of the well UL is shifted from the temperature set at the present stage to the temperature that should be set at the next stage (the time period of a temperature gradient). As the heating assist substrate 13, e.g. a Peltier device is used.

The light emission substrate 14 is a substrate disposed as a layer on the upper side of the reaction substrate 11. On the surface opposed to the reaction substrate 11, of the light emission substrate 14, light source devices LS that emit excitation light for a fluorescent substance such as an intercalator are so disposed as to each correspond to a respective one of the wells UL. As the light source device LS, e.g. a light emitting diode (LED) is used.

The excitation light transmitting substrate 15 is a substrate disposed as an intermediate layer between the reaction substrate 11 and the light emission substrate 14. The excitation light transmitting substrate 15 allows transmission of the excitation light emitted from the light source devices LS therethrough and reflects light other than the excitation light. As this excitation light transmitting substrate 15, e.g. a dichroic mirror is used.

On the surface facing the light emission substrate 14, of this excitation light transmitting substrate 15, light receiving devices LDB that receive scattered light of the excitation light emitted from the light source devices LS are disposed at the periphery of the positions corresponding to the optical axes of the light source devices LS on the light emission substrate 14.

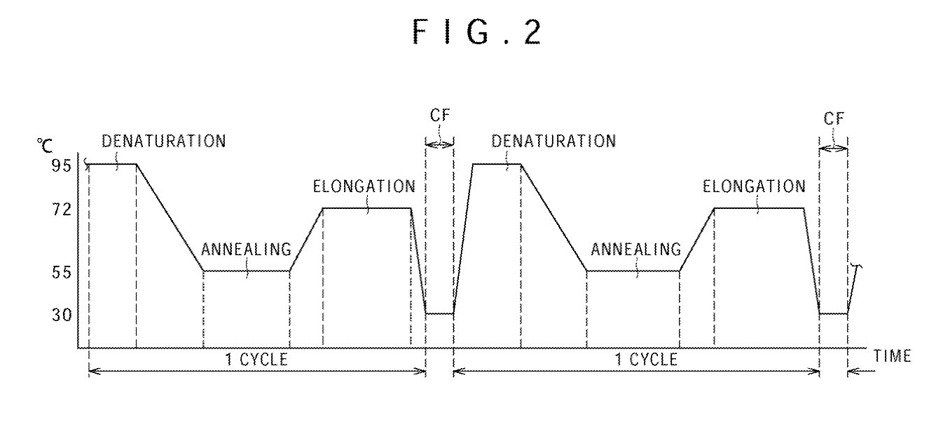

A light amount controller 30 is connected to the light source devices LS on the light emission substrate 14 and the light receiving devices LDB on the excitation light transmitting substrate 15. In this real-time PCR apparatus 1, the next amplification cycle is not immediately started in response to the end timing of one amplification cycle, but as shown in FIG. 2, a calibration period CF in which the amount of light of the light source device LS is adjusted is provided from the end timing of each amplification cycle. In each calibration period CF, the light amount controller 30 individually controls the amount of light of the light source device LS corresponding to the well UL so that the amount of scattered light received by each light receiving device LDB may be constant.

Due to this feature, this real-time PCR apparatus 1 allows the amounts of excitation light reaching the respective wells UL to be kept constant even in the presence of not only variation in the manufacturing of the respective light source devices LS but also change in the light source devices LS over time. Therefore, this real-time PCR apparatus 1 allows enhancement in the detection accuracy so that the amount of fluorescence arising from the excitation by the excitation light may reflect the amount of nucleic acid itself in each well UL.

The fluorescence transmitting substrate 16 is a substrate disposed as an intermediate layer between the heating substrate 12 and the heating assist substrate 13. The fluorescence transmitting substrate 16 allows transmission of fluorescence of the fluorescent substance excited by the excitation light therethrough and reflects light other than the fluorescence. On the surface facing the heating assist substrate 13, light receiving devices LDA that receive the fluorescence arising from the excitation in the wells UL are so disposed as to each correspond to a respective on the wells UL.

A nucleic acid amount calculator 40 is connected to the light receiving devices LDA. The nucleic acid amount calculator 40 calculates the nucleic acid amount of the target nucleic acid dependent on the amount of fluorescence received by each light receiving device LDA on a stage-by-stage basis. Furthermore, the nucleic acid amount calculator 40 acquires the amounts of scattered light, obtained in the present and previous calibration periods. If these amounts of scattered light have a difference, the nucleic acid amount calculator 40 adjusts the nucleic acid amount in the well UL associated with the light source device LS that causes this difference in the amount of scattered light, depending on the difference. This feature allows the nucleic acid amount calculator 40 to equalize the amount of target nucleic acid on the basis of the amount of excitation light even in the presence of change in the light source device LS over time.

In addition to the above-described configuration, this real-time PCR apparatus 1 has a primer evaluation index submitter 50 that submits the index for evaluation of a primer based on the transition (amplification curve) of the amount of fluorescence in the case in which the temperature that should be given at the annealing stage differs.

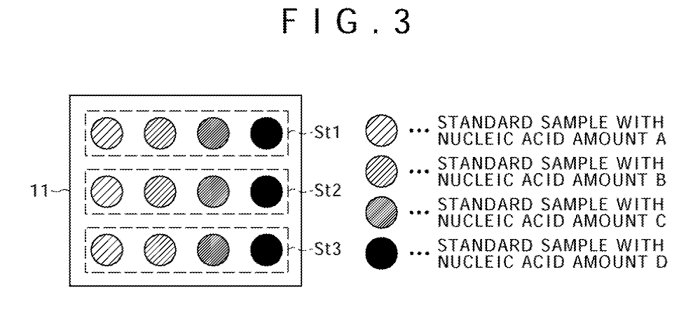

In the case of making the primer evaluation index submitter 50 acquire the evaluation index of a primer, as shown in FIG. 3 conveniently, as many sets St of standard samples obtained by diluting the target nucleic acid that should be employed as the standard in a stepwise manner (hereinafter, the set will be referred to also as the sample set) as the number of temperature conditions that should be made different from each other are prepared in the wells UL. In these wells UL, various substances necessary for the amplification, such as the primer as the evaluation subject, are given.

This real-time PCR apparatus 1 can individually adjust the temperature of each well UL, and therefore the temperature that should be given at the annealing stage can be made different for each sample set St. Therefore, this real-time PCR apparatus 1 allows the primer evaluation index submitter 50 to simultaneously acquire, with one reaction substrate 11, the transitions (amplification curves) of the amount of fluorescence in the case in which the temperature that should be given at the annealing stage differs.

[1-2. Configuration of Primer Evaluation Index Submitter]

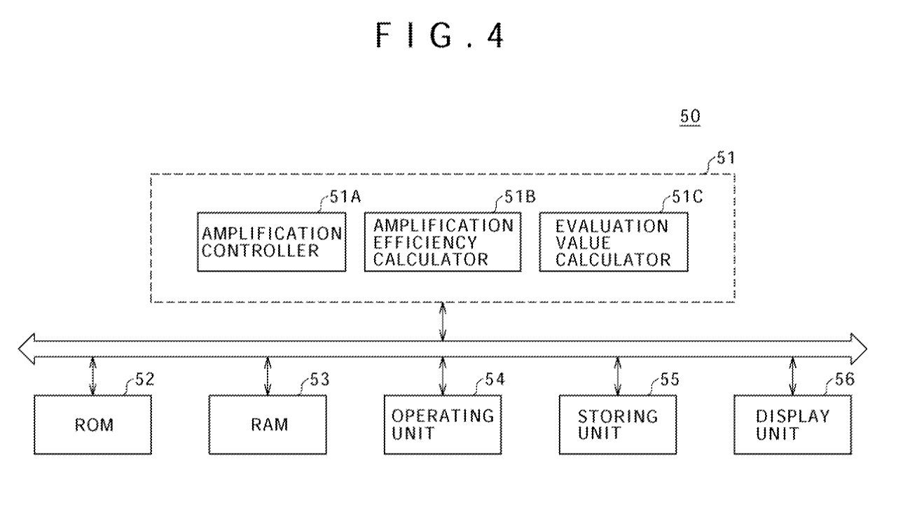

The primer evaluation index submitter 50 will be described below. In FIG. 4, the schematic configuration of the primer evaluation index submitter 50 is shown. This primer evaluation index submitter 50 is configured by connecting various kinds of hardware to a central processing unit (CPU) 51 that is responsible for control of the whole of the primer evaluation index submitter 50.

Specifically, a read only memory (ROM) 52, a random access memory (RAM) 53 serving as the work memory for the CPU 51, an operating unit 54, a storing unit 55, and a display unit 56 are connected to the CPU 51. In this ROM 52, a program for generating data of the evaluation index of a primer (hereinafter, it will be referred to also as the primer evaluation index submission program) is stored.

If the primer evaluation index submission program stored in the ROM 52 is expanded in the RAM 53, the CPU 51 functions as an amplification controller 51A, an amplification efficiency calculator 51B, and an evaluation value calculator 51C in accordance with the primer evaluation index submission program.

The amplification controller 51A decides the same temperature for each of the sample sets St as the target temperatures at the denaturation stage and the elongation stage. In addition, the amplification controller 51A decides temperatures different for each sample set St as the target temperatures at the annealing stage.

As the method for deciding the target temperature, e.g. a method of deciding the temperature through selection among the temperatures prescribed in the primer evaluation index submission program or a method of deciding the temperature through input from the operating unit 54 is employed.

The amplification controller 51A displays, on the display unit 56, e.g. an input screen for the initial nucleic acid amount (concentration) of each standard sample in the sample set St and acquires the initial nucleic acid amounts from the operating unit 54.

If the amplification controller 51A decides the target temperatures at the respective amplification stages for the sample set St, the amplification controller 51A instructs the temperature controller 20 to set the decided target temperatures. Subsequently, the amplification controller 51A drives the temperature controller 20 and the light amount controller 30 to start amplification treatment along the amplification cycle.

In response to the start of the amplification treatment by the amplification controller 51A, a signal indicating the amounts of amplification (the amounts of fluorescence) is given to the amplification efficiency calculator 51B from the light receiving devices LDA (FIG. 1) corresponding to the wells UL in which the respective standard samples in the sample set St are disposed.

The amplification efficiency calculator 51B detects the number of amplification cycles repeated until certain fluorescence intensity is obtained based on the amount of amplification (the amount of fluorescence) given from the light receiving device LDA and the number of amplification cycles counted by the amplification controller 51A.

Furthermore, the amplification efficiency calculator 51B calculates the amplification efficiency of the standard sample for each sample set St. Specifically, the amplification efficiency is calculated from the slope of a standard curve obtained by plotting, on the abscissa, the initial nucleic acid amounts of the respective standard samples in the sample set St acquired by the amplification controller 51A and plotting, on the ordinate, the numbers of amplification cycles repeated until certain fluorescence intensity is obtained.

A simple description will be made below about the relationship between the slope of the standard curve and the amplification efficiency. In general, it is theoretically considered that a template DNA is amplified by a factor of two through one cycle in the PCR. If the initial amount of template DNA is defined as [DNA]₀, the amplification efficiency is defined as e, and the number of amplification cycles is defined as C, the amount of DNA is represented by the following equation.

[DNA]=[DNA]₀(1+e)^{C} (1)

When the amplification efficiency e is 1, the number inside the parenthesis is 2 in this Equation (1) and therefore the template DNA is amplified by a factor of two through one cycle.

Taking the logarithm of both sides of Equation (1) provides the following equation

$$\begin{matrix}log [ DNA ] = {log [ DNA ]}_{0} + {log ( 1 + e )}^{C} \\ = {log [ DNA ]}_{0} + C log ( 1 + e )\end{matrix} \qquad ( 2 )$$

If the cycle through which certain fluorescence intensity is achieved is defined as Ct, the following equation is obtained.

$$Ct = \frac{{log [ DNA ]}_{t} - {log [ DNA ]}_{0}}{log ( 1 + e )} \qquad ( 3 )$$

The cycle Ct is represented as a function of log [DNA]₀ and the slope of the standard curve is −1/log(1+e). If the amplification efficiency is 100% (e=1), the slope of the standard curve is −3.32. If the amplification efficiency is 85% (e=0.85), the slope is −3.74. That is, the slope of the standard curve and the amplification efficiency are in such a relationship that a steeper slope of the standard curve indicates lower amplification efficiency. With another expression, the amplification efficiency e can be represented by the following equation.

e=10^{-1/slope}−1 (4)

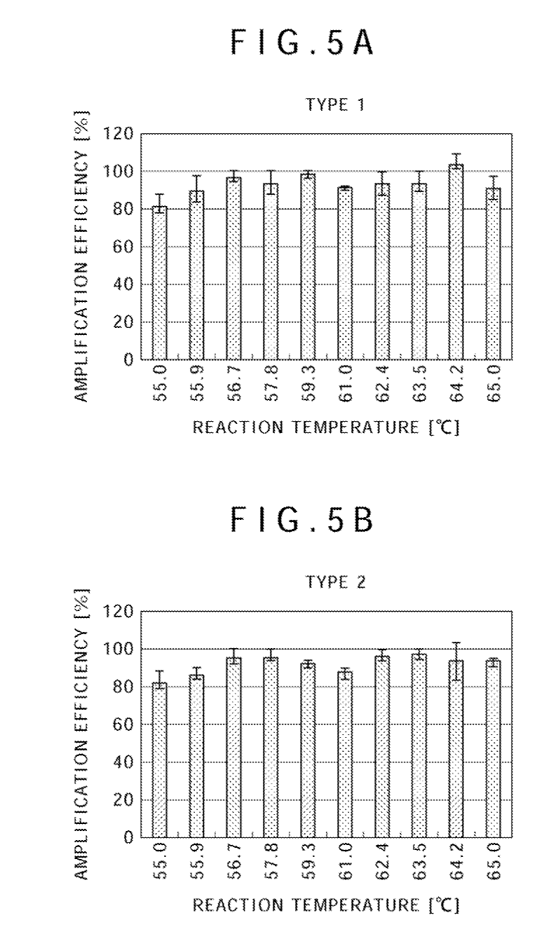

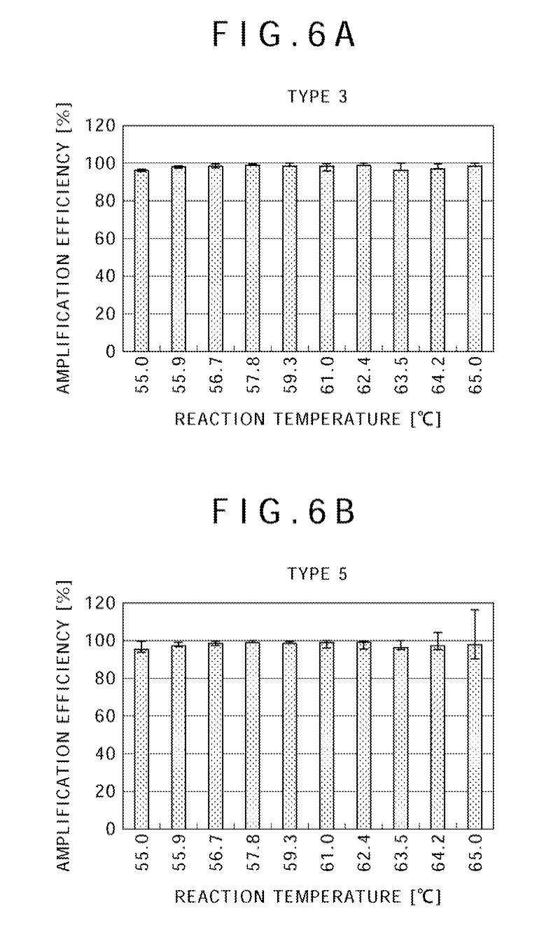

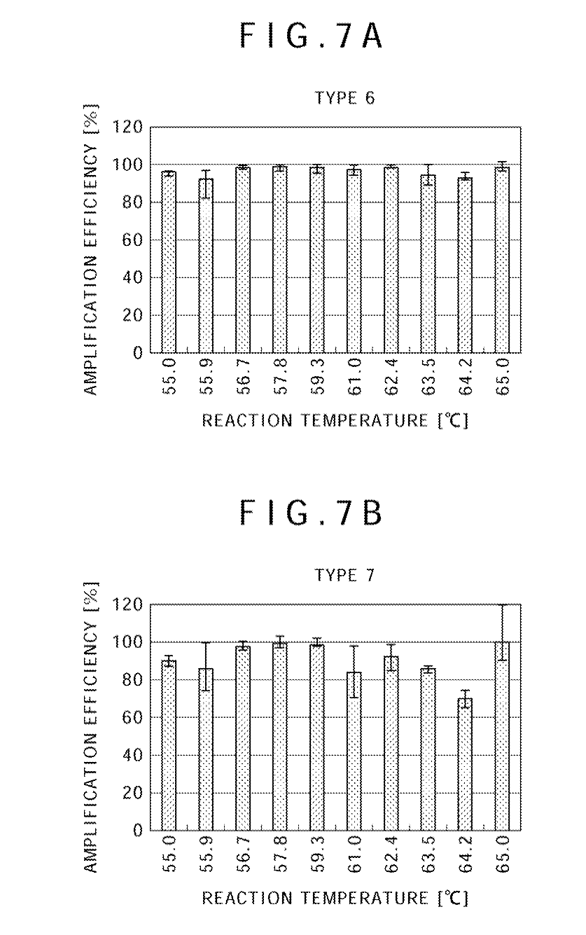

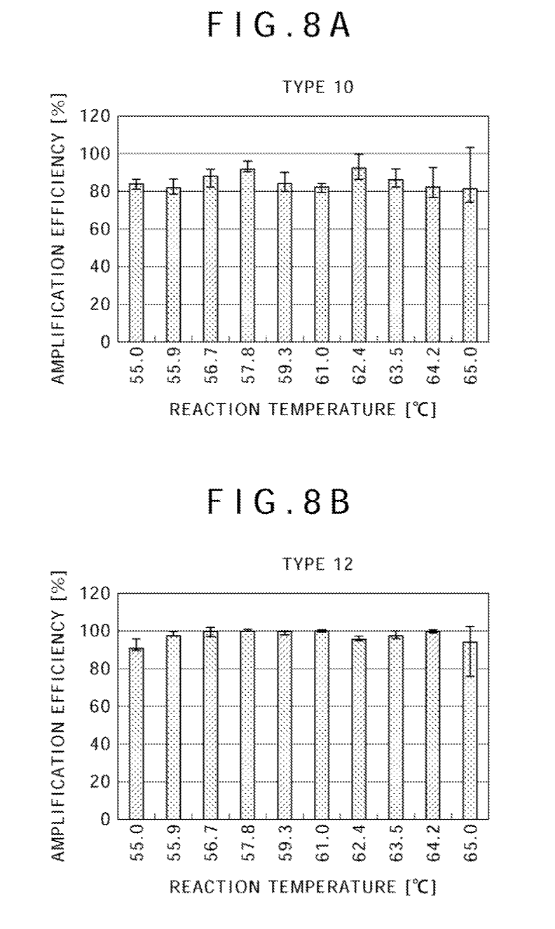

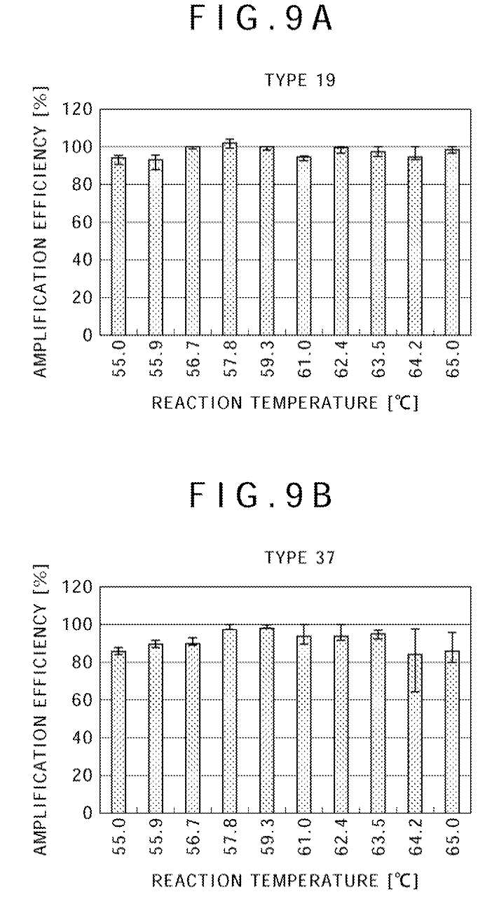

As experimental results, the amplification efficiency in the case in which the temperature that should be given at the annealing stage differs is shown in FIGS. 5A to 9B for each type of an adenovirus. In this experiment, the primer was designed on a type-by-type basis. Furthermore, as shown in FIGS. 5A to 9B, the target temperatures at the annealing stage for the sample sets St were set to 55.0° C., 55.9° C., 56.7° C., 57.8° C., 59.3° C., 61.0° C., 62.4° C., 63.5° C., 64.2° C., and 65.0° C. The conditions other than the primer and the target temperature at the annealing stage were the same.

FIGS. 5A to 9B show that e.g. the primer for type 3 exhibits equivalent amplification efficiency irrespective of the temperature change whereas e.g. the primer for type 7 exhibits amplification efficiency that changes depending on the temperature change.

The evaluation value calculator 51C calculates the variation degree of the amplification efficiency of each sample set St as the index for evaluating the primer. As this variation degree, specifically e.g. the standard deviation, the variance, or the average deviation is used.

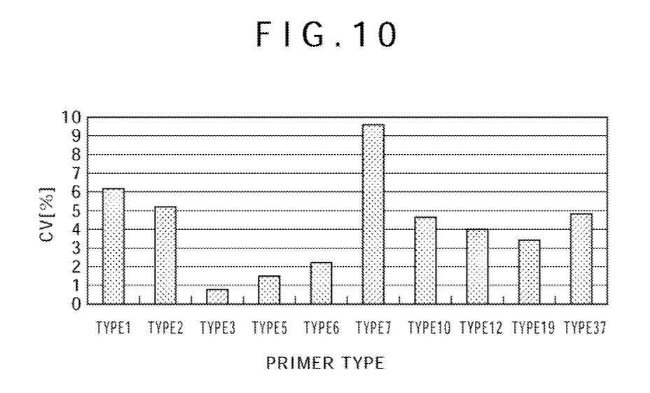

FIG. 10 shows the variation degrees of the amplification efficiency at the respective temperatures, shown in FIGS. 5A to 9B for each type. As is apparent from FIG. 10, the primer for type 3 has the lowest temperature dependence and thus can be regarded as a favorable primer. On the other hand, the primer for type 7 has the highest temperature dependence and thus can be regarded as a poor primer.

If the evaluation value calculator 51C calculates the variation degree of the amplification efficiency of each sample set St, the evaluation value calculator 51C compares the variation degree with the reference value for the quality evaluation of the primer, with respect to the variation degree (hereinafter, the reference value will be referred to also as the variation threshold). In addition, if the variation degree as the calculation subject is equal to or lower than the variation threshold, the evaluation value calculator 51C notifies the user of that the primer is a favorable primer, the variation degree as the calculation subject, and the variation threshold by image displaying or audio.

In contrast, if the variation degree as the calculation subject is higher than the variation threshold, the evaluation value calculator 51C notifies the user of that the primer is a poor primer, the variation degree as the calculation subject, and the variation threshold by image displaying or audio.

As the form of the image displaying, e.g. a form in which a line indicating the variation threshold is shown in a graph indicating the variation degree by a bar is employed. It is also possible to display a graph indicating the relationship between the temperatures made different from each other and the amplification efficiency in addition to the graph indicating the variation degree by a bar.

The evaluation value calculator 51C in this embodiment can switch the variation threshold depending on the use purpose of the result of the propagation reaction by use of the primer as the evaluation subject.

Specifically, for example, a first variation threshold that should be used for general purpose is set. In addition, as values smaller than this reference threshold, a second variation threshold that should be used for comparison purpose in pharmacological experiment and a third variation threshold that should be used for diagnostic purpose in clinical practice are set. The user selects one threshold among these thresholds.

Due to this feature, the evaluation value calculator 51C allows the user to design a primer having temperature resistance at a certain level depending on the degree of importance of the primer design (i.e. the use purpose of the result of the propagation reaction), even with consideration of the given design time.

[1-3. Procedure of Primer Evaluation Index Submission Processing]

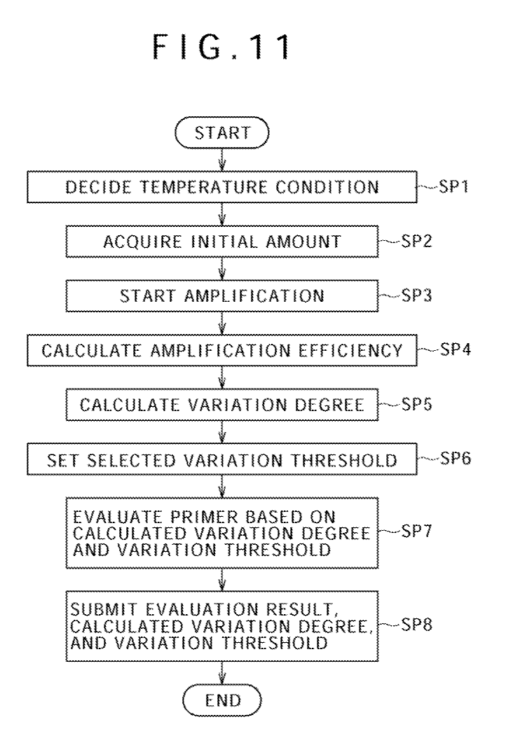

The procedure of primer evaluation index submission processing will be described below with use of a flowchart shown in FIG. 11.

Specifically, if an instruction to submit the evaluation index for a primer is given from the operating unit 54 to the CPU 51 for example, the CPU 51 starts the procedure of the primer evaluation index submission processing and moves to a first step SP1. In this first step SP1, for each sample set St, the CPU 51 decides the target temperature at the annealing stage for the sample set and moves to a second step SP2.

In this second step SP2, the CPU 51 acquires the initial amounts (concentrations) of the respective standard samples in the sample set St and moves to a third step SP3. In this third step SP3, the CPU 51 starts amplification treatment for the respective sample sets St under the condition of the target temperatures decided in the first step SP1, and moves to a fourth step SP4.

In this fourth step SP4, the CPU 51 calculates the amplification efficiency for each sample set St and moves to a fifth step SP5. In this fifth step SP5, the CPU 51 calculates the variation degree of the amplification efficiency of the respective sample sets St and moves to a sixth step SP6.

In this sixth step SP6, the CPU 51 allows selection of the variation threshold to set the selected variation threshold, and moves to a seventh step SP7. In this seventh step SP7, the CPU 51 evaluates the primer based on the variation degree calculated in the step SP5 and the variation threshold set in the step SP6, and moves to an eighth step SP8.

In this eighth step SP8, the CPU 51 notifies the user of the result of the primer evaluation, the variation degree as the calculation subject, and the variation threshold by image displaying or audio. After the notification, the CPU 51 ends this procedure of the primer evaluation index submission processing.

In this manner, the CPU 51 executes the primer evaluation index submission processing in accordance with the primer evaluation index submission program.

[1-4. Advantageous Effects and so on]

In the above-described configuration, the real-time PCR apparatus 1 acquires a signal indicating time change in the amplification amount obtained when amplification is so performed that the temperature condition at the stages other than the annealing stage is fixed for each of the sample sets St (see FIG. 3) prepared for the number of temperature conditions that should be made different from each other.

Furthermore, the real-time PCR apparatus 1 acquires the initial amounts (concentrations) of the standard samples that are included in the sample set St and obtained by dilution in a stepwise manner, and obtains the amplification efficiency for each temperature condition based on the initial amount and the time change in the amplification amount (see FIGS. 5A to 9B).

In addition, the real-time PCR apparatus 1 calculates the variation degree of the amplification efficiency obtained for each temperature condition (see FIG. 10), and submits the calculated variation degree and the reference value (variation threshold) for the quality evaluation of the primer, set with respect to the variation degree.

This real-time PCR apparatus 1 can detect the temperature dependence of the primer because it obtains the variation degree of the amplification efficiency obtained when amplification is so performed that the temperature condition at the stages other than the annealing stage is fixed. In addition to this, the real-time PCR apparatus 1 allows the design of a primer having temperature resistance at a certain level because it submits this variation degree together with the variation threshold.

This real-time PCR apparatus 1 has the temperature controller 20 (FIG. 1) that individually controls the amount of heat that should be given to the plural wells UL depending on the temperature set for the wells UL. For this temperature controller 20, the real-time PCR apparatus 1 decides the annealing-stage temperature that should be set for the wells UL in which the target nucleic acid in the sample set is disposed for each sample set St.

Furthermore, the real-time PCR apparatus 1 acquires the signal indicating time change in the amplification amount obtained when amplification is so performed that the temperature condition at the stages other than the annealing stage is fixed, from the light receiving device LDA (FIG. 1) allocated to the corresponding well UL.

Therefore, this real-time PCR apparatus 1 can simultaneously acquire, with one reaction substrate 11, time changes in the amplification amount (amplification curves) in the case in which the temperature condition that should be given at the annealing stage differs. As a result, this real-time PCR apparatus 1 can significantly enhance the efficiency of calculation of the variation degree of the amplification efficiency.

Moreover, this real-time PCR apparatus 1 switches the variation threshold depending on the use purpose of the result of the propagation reaction by use of the primer as the evaluation subject.

Therefore, the real-time PCR apparatus 1 can set the variation threshold strictly or loosely depending on the degree of importance of the primer design (i.e. the use purpose of the result of the propagation reaction), such as general purpose, comparison purpose in pharmacological experiment, and diagnostic purpose in clinical practice. As a result, this real-time PCR apparatus 1 allows the user to design a primer having temperature resistance at a certain level depending on the degree of importance of the primer design, even with consideration of the given design time.

According to the above-described configuration, because of the characteristic that the variation degree of the amplification efficiency in the case in which the temperature condition at the annealing stage of the propagation cycle is made different is submitted in addition to the variation threshold, the real-time PCR apparatus 1 that allows the design of a primer having temperature resistance at a certain level can be realized.

### 2. Other Embodiments

The target temperature at the denaturation stage and the elongation stage may be a fixed value prescribed in advance although the target temperature is decided through selection among temperatures prescribed in the program or decided through input from the operating unit 54 in the above-described embodiment.

Furthermore, in the above-described embodiment, as the acquisition form in which the signal indicating time change in the amplification amount in the case in which the temperature condition at the annealing stage of the propagation cycle is made different is acquired, the form in which the signal is acquired by using the same reaction substrate 11 is employed. However, the acquisition form is not limited to this embodiment.

For example, a form in which the signal is acquired by using the reaction substrates 11 different for each temperature condition may be employed. Alternatively, for example, it is possible to employ a form in which the signal is acquired from a data storing medium in which the signal indicating time change in the amplification amount in the case in which the temperature condition at the annealing stage of the propagation cycle is made different is stored.

Examples of the data storing medium include package media such as a flexible disk, a compact disk-read only memory (CD-ROM), and a digital versatile disk (DVD), and other media such as a semiconductor memory and a magnetic disk in which data is stored temporarily or permanently. As the method for acquiring data from these data storing media, it is also possible to utilize a wired or wireless communication medium such as a local area network, the Internet, or digital satellite broadcasting.

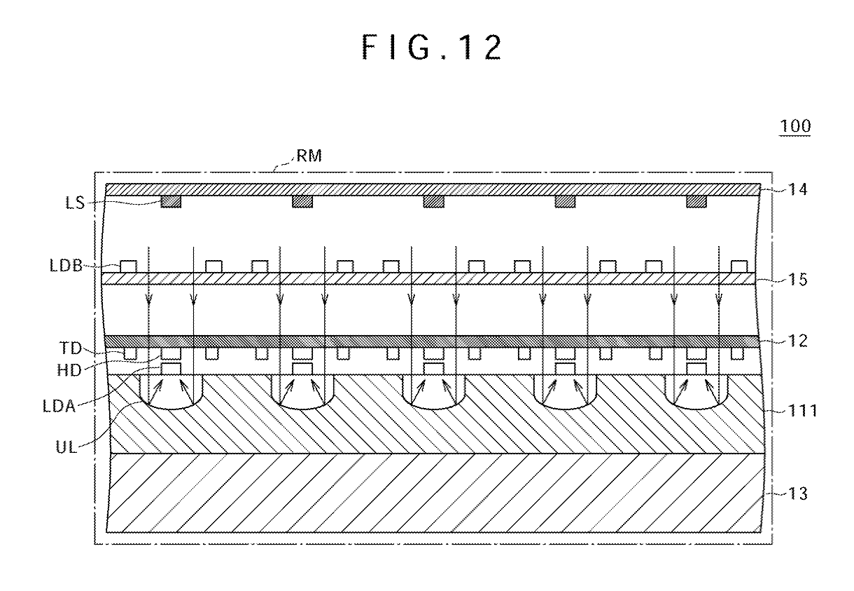

In addition, in the above-described embodiment, so-called transmissive-type apparatus is employed as the real-time PCR apparatus 1. However, instead of this, for example as shown in FIG. 12 in which the same parts as those in FIG. 1 are given the same numerals, so-called reflective-type real-time PCR apparatus 100 may be employed.

In a reaction substrate 111 in this real-time PCR apparatus 100, plural wells UL whose sidewall bottoms are formed into a round shape. Furthermore, the light receiving devices LDA are so disposed on one surface of the reaction substrate 111 as to correspond to these wells UL. Excitation light emitted from each light source LS is introduced via the excitation light transmitting substrate 15 to the corresponding well UL formed in the reaction substrate 111. In each well UL, fluorescence of the fluorescent substance excited by the excitation light is reflected by the wall surface of the well UL. The reflected fluorescence is incident on the light receiving device LDA formed on one surface of the reaction substrate 111.

Also when such reflective-type real-time PCR apparatus 100 is employed, the same advantageous effects as those by the above-described embodiment can be achieved.

The embodiments can be utilized in the bioindustry encompassing gene experiment, creation of a medicine, follow-up of a patient, and so on.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A primer evaluation method comprising:
(a) acquiring a signal indicating time change in an amplification amount obtained when sample sets prepared for a number of temperature conditions that should be made different from each other at an annealing stage in units of target nucleic acids diluted in a stepwise manner are so amplified that a temperature condition at a stage other than the annealing stage is fixed;
(b) acquiring a signal indicating initial amounts of the target nucleic acids diluted in a stepwise manner;
(c) obtaining amplification efficiency for each of the temperature conditions based on the time change in the amplification amount and the initial amount, and calculating a variation degree of the amplification efficiency; and
(d) submitting the variation degree and a reference value for quality evaluation of a primer, set with respect to the variation degree.

2. The primer evaluation method according to claim 1, further comprising
determining an annealing-stage temperature that should be set for containers in which the target nucleic acids in the sample set are disposed for each of the sample sets, for a controller that individually controls an amount of heat that should be given to a plurality of containers used as a place of amplification reaction of the target nucleic acids depending on a temperature set for a corresponding container, wherein
in the step (a), the signal indicating the time change in the amplification amount is acquired from a light receiving device allocated to the container.

3. The primer evaluation method according to claim 2, further comprising
switching the reference value depending on use purpose of a result of propagation reaction by use of a primer as an evaluation subject.

4. The primer evaluation method according to claim 3, wherein in (d),
the variation degree calculated in (c) is compared with the reference value, and
an evaluation result indicating that a primer used in the propagation is favorable is also submitted if the variation degree is equal to or lower than the reference value, and an evaluation result indicating that a primer used in the propagation is poor is also submitted if the variation degree is higher than the reference value.

5. A primer evaluation computer program product including executable instructions that when executed by a processor perform steps for
acquiring, from an apparatus capable of propagating a nucleic acid or a storage medium, a signal indicating a time change in an amplification amount obtained when sample sets prepared for a number of temperature conditions that should be made different from each other at an annealing stage in units of target nucleic acids diluted in a stepwise manner are so amplified that a temperature condition at a stage other than the annealing stage is fixed;
acquiring a signal indicating initial amounts of the target nucleic acids diluted in a stepwise manner;
obtaining amplification efficiency for each of the temperature conditions based on the time change in the amplification amount and the initial amount, and calculating a variation degree of the amplification efficiency; and
submitting the variation degree and a reference value for quality evaluation of a primer, set with respect to the variation degree.

6. A real-time polymerase chain reaction apparatus comprising:
a heat source device allocated to a plurality of containers formed as a place of amplification reaction of a nucleic acid in a substrate;
control means for individually controlling an amount of heat of the heat source device depending on a temperature set for a corresponding container;
determination means for determining, for each of sample sets prepared for a number of temperature conditions that should be made different from each other in units of target nucleic acids diluted in a stepwise manner, an annealing-stage temperature that should be set for the containers in which the target nucleic acids in the sample set are disposed;
initial amount acquiring means for acquiring a signal indicating initial amounts of the target nucleic acids diluted in a stepwise manner;
amplification amount acquiring means for acquiring, from a plurality of light receiving devices allocated to the containers, a signal indicating an amplification amount obtained when the sample sets are so amplified that a temperature condition at a stage other than an annealing stage is fixed;
calculating means for obtaining amplification efficiency for each of the temperature conditions based on time change in the amplification amount and the initial amount, and calculating a variation degree of the amplification efficiency; and
submitting means for submitting the variation degree and a reference value for quality evaluation of a primer, set with respect to the variation degree.
